SEQUENCE LISTING (9 sequences)

SEQ ID NO: 1 - DNA, 19 bp, artificial sequence
  FW primer for C785T at pos 10,638-10,657
tcgtcctcatcttcctggt

SEQ ID NO: 2 - DNA, 19 bp, artificial sequence
  FM primer for C785T at pos 10,638-10,657
tcgtcctcatcttcctggt

SEQ ID NO: 3 - DNA, 20 bp, artificial sequence
  R primer for C785T at pos 10,638-10,657
agtccatggcgaaacttctg

SEQ ID NO: 4 - DNA, 19 bp, artificial sequence
  FW primer for T1208C at pos 11,061-11,080
ccattgtggtcacaggatg

SEQ ID NO: 5 - DNA, 19 bp, artificial sequence
  FM primer for T1208C at pos 11,061-11,080
ccattgtggtcacaggatg

SEQ ID NO: 6 - DNA, 20 bp, artificial sequence
  R primer for T1208C at pos 11,061-11,080
tgcagagctgtctcactgga

SEQ ID NO: 7 - DNA, 26 bp, artificial sequence
  RW primer for G1440A at pos 11,338-11,312
gtatttttagtagagacagggtttga

SEQ ID NO: 8 - DNA, 26 bp, artificial sequence
  RM primer for G1440A at pos 11,338-11,312
gtatttttagtagagacagggtttga

SEQ ID NO: 9 - DNA, 19 bp, artificial sequence
  F primer for G1440A at pos 11,338-11,312
cctcaccccttgccataat

(19) United States
(12) Patent Application Publication
(10) Pub. No.: US 2005/0250132 A1
(43) Pub. Date: Nov. 10, 2005

(54) Real-time polymerase chain reaction-based genotyping assay for chemokine receptor (CXCR2) single nucleotide polymorphism

(76) Inventors: Bernd Melbohm, Memphis, TN (US); Manish Gupta, Memphis, TN (US); Charles R. Yates, Collierville, TN (US)

Correspondence Address: Benjamin Aaron Adler;ADLER & ASSOCIATES, 8011 Candle Lane, Houston, TX, 77071, US

(21) Appl. No.: 11/066,586

(22) Filed: Feb. 25, 2005

Related U.S. Application Data
(60) Provisional application No. 60/547,480, filed on Feb. 25, 2004.

(51) Int. Cl. C12Q 1/68; C07H 21/04

(52) U.S. Cl. 435/6; 536/24.3

(57) ABSTRACT

The present invention provides fluorescence-based real-time PCR assays for the rapid detection of chemokine receptor single nucleotide polymorphisms (SNPs). The genotyping assay can be used to detect SNPs of human chemokine receptor (CXCR2) single nucleotide polymorphisms T1208C, C785T and G1440A.

### CROSS REFERENCE TO RELATED APPLICATION

This non-provisional application claims benefit of provisional application U.S. Ser. No. 60/547,480 filed Feb. 25, 2004, now abandoned.

### BACKGROUND OF THE INVENTION

1. Field of the Invention

The present invention relates generally to the field of single nucleotide polymorphism genotyping. More specifically, the present invention provides a real-time polymerase chain reaction-based genotyping assay for the detection of chemokine receptor (CXCR2) single nucleotide polymorphisms.

2. Description of the Related Art

Chronic obstructive pulmonary disease (COPD) is characterized by decreased expiratory flow rates, increased pulmonary resistance and hyperinflation of the lung. It is a major medical problem and a leading cause if morbidity and mortality among the adult population. In the U.S., COPD affects >16 million people, accounts for 13% of hospitalizations and is the fourth leading cause of death. Although cigarette smoking is the major risk factor, only 10-20% smokers develop symptomatic COPD. Numerous epidemiological studies provide compelling evidence that genetic factors influence the development of COPD. Genetic risk factors for COPD may include the inherited deficiency of α1-antitrypsin in certain individuals, which is relatively uncommon and explains only a very small proportion (<1%) of COPD cases (Poller et al., 1990).

The associations between COPD and polymorphisms in several other genes suspected to be involved in COPD pathogenesis have been studied. These include α1-antichymotrypsin (Sandford, A. J. et al., 1998; Ishii, T. et al., 2000; Poller, W. et al., 1993; Benetazzo, M. G. et al., 1999), microsomal epoxide hydrolase (Cheng, S. L. et al., 2004; Yim, J. J. et al., 2000; Yoshikawa, M. et al., 2000; Harrison, D. J. et al., 1997; Rodriguez, F. et al., 2002), vitamin D-binding protein (Laufs, J. et al., 2004; Schellenberg, D. et al., 1998), glutathione-S-transferase (Harrison, D. J. et al., 1997; Yim, J. J. et al., 2002; Ishii, T. et al., 1999), cytochrome P4501A1 (Dialyna, I. et al., 2003), immunoglobulin-A (Ruse, C. E. et al., 2003; van der Pouw Kraan, T. C. et al., 2002), matrix metalloproteinases (Joos, L. et al., 2002; Minematsu, N. et al., 2001), tumor necrosis factor-α (Ishii, T. et al., 2000; Higham, M. A. et al., 2000; Keatings, V. M. et al., 2000; Teramoto, S. and Ishii, T., 2001; Sakao, S. et al., 2001) and beta-2 adrenergic receptor (Ho, L. I. et al., 2001; Joos, L. et al., 2003). Results of some of these studies have been inconsistent and in most cases these polymorphisms were found not to be associated with the frequency and severity of COPD. In COPD, it is likely that multiple genes are operating and genetic susceptibility may depend on the coincidence of several gene polymorphisms acting together. The search and identification of genetic factors that contribute to the progression of COPD will likely clarify and deepen the understanding of the pathogenesis of COPD and will lead to a more specific and successful treatment.

Interleukin-8 (IL-8), a member of the CXC chemokine family acts as a potent activator and chemoattractant of neutrophils (Holmes et al., 1991; Mollereau, et al., 1993; Morris et al., 1992; Sprenger et al., 1994; Ahuja et al., 1994). Cellular activities of IL-8 are mediated by two receptors, CXCR1 (IL8RA) and CXCR2 (IL8RB), which are encoded by genes located on chromosome2q34-q35 (Holmes et al., 1991; Mollereau, C. et al., 1993). The IL-8Rs are members of the G-protein-coupled family of receptors, which feature seven transmembrane domains. The two receptors have 78% homology in protein amino acid sequence and bind IL-8 with similar affinity (Morris et al., 1992). While CXCR1 binds only two CXC chemkines, IL-8 and granulocyte chemotactic protein (GCP)-2, CXCR2 also binds with high affinity to other CXC chemokines for example GROα, GROβ, GROγ and neutrophil-activating peptide 2 (Baggiolini, M., 1998) and is involved in the chemotaxis of immune cells. The CXCR2 gene consists of 11 exons. The open reading frame is encoded entirely by a single exon, exon 11. The remaining exons are alternatively spliced, giving rise to seven distinct messenger RNA (mRNA) variants (Sprenger et al., 1994; Ahuja, S. K. et al., 1994). The mapped susceptibility loci for several human disorders, such as rheumatoid arthritis, systemic lupus erythematosus, insulin-dependent diabetes mellitus, and juvenile amyotrophic lateral sclerosis include chromosome 2q35 (Kato, H. et al., 2000). Hence, polymorphisms in this region may have relevance for susceptibility to and pathogenesis of these disorders. Further several studies point to a crucial role for IL8 and/or its receptors in the pathogenesis of COPD.

Three novel single nucleotide polymorphisms occurring in this gene have been identified (Renzoni et al, 2000). These include single nucleotide polymorphism of the CXCR2 gene at nucleotide position 785 (C→T) results in silent mutation with codon change from CTC (leucine) to CTT (leucine), whereas the polymorphisms at positions 1208 (T→C), and 1440 (G→A) occur in the non-coding region of the CXCR2 gene (corresponding to positions 10657, 11080 and 11312 of sequence M99412, respectively). All of these polymorphisms have the potential of altering mRNA processing, stability or translation.

It has been suggested that genetic variants of CXCR2 may provide valuable information for the pathogenesis of and susceptibility to chronic inflammatory conditions involving neutrophil recruitment, especially in rheumatoid and respiratory diseases (Kato et al, 2000, Renzoni et al, 2000, Barnes, 1999, Hamajima et al, 2002). Investigation of the distribution of these polymorphisms in systemic sclerosis and cryptogenic fibrosing alveolitis revealed a strong linkage between the 785C, 1208T and 1440G alleles. There was a significant increase in the frequency of homozygous 785T and 1208C genotypes in systemic sclerosis patients compared to a control group. The allele frequency for 785C, 1208T and 1440G in Caucasian control subjects was 0.48, 0.44 and 0.44 respectively.

Real-time polymerase chain reaction (PCR) has recently evolved as a fast and inexpensive methodology for allelic discrimination assays. To facilitate clarification of the association of single nucleotide polymorphisms (SNPs) in the candidate chemokine receptor (CXCR2) gene with the susceptibility to chronic inflammatory conditions involving neutrophil recruitment, especially in rheumatoid and respiratory diseases, a rapid and robust real-time PCR-based genotyping assay to detect the single nucleotide polymorphisms of chemokine receptor (CXCR2) at nucleotide position 1208 (T→C), 785 (C→T) and 1440 (G→A) would be highly desirable.

The prior art is deficient in a rapid and robust real-time PCR-based genotyping assay to detect the single nucleotide polymorphisms of the chemokine receptor CXCR2. The present invention fulfills this longstanding need and desire in the art.

### SUMMARY OF THE INVENTION

The present invention provides a real-time polymerase chain reaction (PCR)-based method to detect single nucleotide polymorphisms of the chemokine receptor (CXCR2). Discrimination between wild type and mutant alleles was achieved using PCR amplification of specific alleles modified to prevent non-Watson Crick base pairing (Okimoto & Dodgson, 1996; Sommer et al., 1992; Bottema et al., 1993; Newton et al., 1989). Two key nucleotide mismatches are required for allelic discrimination. The first nucleotide difference between primers used to discriminate between wild type and mutant alleles was located at the 3′ terminal base. However, a single base pair difference at the 3′ end of the primer is insufficient, in most cases, to achieve allelic discrimination. An additional internal nucleotide mismatch (typically within 5 base pairs of the 3′ end) is required for specific amplification of either the wild-type or mutant allele. Thus, a second nucleotide mismatch located three bases from the 3′ end for both the wild-type and mutant-specific primers was included to generate an internal primer/template mismatch that prevents amplification of the nonmatching primer.

The present invention provides a genotyping assay to detect single nucleotide polymorphism (SNP) of chemokine receptor (CXCR2) gene, where the single nucleotide polymorphism is a T1208C, C785T and a G1440A polymorphism. T1208C results in polymorphism in the non-coding region of CXCR2 gene. PCR reactions for genotyping T1208C using allele-specific primers were conducted in separate tubes. PCR amplification was monitored by Smart Cycler® (Cepheid, Sunnyvale, Calif.) using SYBR Green I, a non-specific double stranded DNA intercalating fluorescent dye. PCR growth curves exceeding the threshold cycle were considered positive. Fluorescence melt-curve analysis was used to corroborate results from PCR growth curves. Using PCR growth curves, the assay disclosed herein accurately determined hetero- and homozygosity for T1208C. Genotype assignments based on PCR growth curve, melt-curve analysis, agarose gel electrophoresis, and direct DNA sequencing results of PCR products were in perfect agreement. Thus, the present invention provides a rapid chemokine receptor (CXCR2) genotyping method that can be used by a person having ordinary skill in this art to assess the contribution of chemokine receptor (CXCR2) single nucleotide polymorphisms to the prevalence and severity of chronic inflammatory conditions. Additionally, the present invention also investigated the association between airflow limitation and CXCR2 polymorphisms by evaluating the database of patients from ‘Health, Aging and Body Composition’ (Health ABC) study, with and without airflow limitation.

In one embodiment, the present invention provides a primer pair for genotyping a single nucleotide polymorphism of human chemokine receptor (CXCR2) gene. This primer pair comprises an allele specific forward primer and a reverse primer or an allele specific reverse primer and a forward primer.

In another embodiment, the present invention provides a method of genotyping a single nucleotide polymorphism of human chemokine receptor (CXCR2) gene. This method comprises extracting DNA from sample of an individual. The DNA is then amplified in separate PCR reactions comprising a wild type allele specific forward primer and a reverse primer and a mutant allele specific forward primer and the reverse primer. Alternatively, the separate PCR reactions comprise a wild type allele specific reverse primer and a forward primer and a mutant allele specific reverse primer and a forward primer. This is followed by identification of the products of the DNA amplification, wherein presence of products amplified by the allele specific forward primers and the reverse primer or the allele specific reverse primer and the forward primer indicate that the individual has the single nucleotide polymorphism of human chemokine receptor (CXCR2) gene.

In yet another embodiment, the present invention provides a kit for genotyping a single nucleotide polymorphism of human chemokine receptor (CXCR2) gene. This kit comprises allele specific forward primers and a reverse primer. Alternatively, the kit may also comprise allele specific reverse primers and a forward primer.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 shows chemokine receptor (CXCR2) T1208C allelic discrimination by real-time analysis using the Smart Cycler. Plot of fluorescence versus cycle number using human genomic DNA obtained from individuals with TT (Panel A), TC (Panel B), or CC (Panel C) genotypes. Interrogation for the presence of either the T or C allele was conducted in physically separate tubes using the common reverse primer 1208R coupled with either the wild type specific primer 1208FW or the mutant-specific primer 1208FM. PCR growth curves that exceed the threshold fluorescence (Cₜ) indicate specific PCR product formation.

FIG. 2 shows melt curve analysis of PCR products using SYBR Green I. Melt curves were converted to melt peaks by plotting the negative first derivative of the fluorescence versus temperature ([-dF/dT]). Plot of [-dF/dT] versus temperature obtained after amplification of TT (Panel A), TC (Panel B), CC (Panel C) genomic DNA using the common reverse primer 1208R coupled with either the wild type specific primer 1208FW or the mutant-specific primer 1208FM. The melt temperature (Tₘ=86° C.) was identical for PCR products formed using either the wild type or mutant-specific primers.

FIG. 3 shows the identification of polymorphisms in nucleic acid 1208 of the chemokine receptor (CXCR2) in three patients by conventional modified allele-specific PCR. An ethidium bromide-stained 2% agarose gel containing PCR fragments (198 bp) was run to confirm real-time PCR results. Odd-numbered lanes contain PCR fragments after amplification with 1208R and 1208FW. Even-numbered lanes contain PCR fragments after amplification with 1208R and 1208FM. PCR products amplified from genomic DNA with different genotypes were loaded as follows: homozygotes T/T (lanes 1 and 2), heterozygotes T/C (lanes 3 and 4), and nullizygotes C/C (lanes 5 and 6). M: Molecular marker which contains a 100-bp DNA ladder.

FIGS. 4A-C show genotype distribution based on CXCR2 polymorphisms in subjects. FIG. 4A shows genotype distribution for polymorphism at position 785 in subjects with airflow limitation (Cases) and in those without airflow limitations (Controls). There was a significant difference in genotype frequencies between the groups (P=0.05 for a 3×2 contingency table). Heterozygosity for the CT genotype was significantly more frequent in cases. FIG. 4B shows genotype distribution for polymorphism at position 1208 in subjects with airflow limitation (Cases) and in those without airflow limitation (Controls). There was a significant difference in genotype frequencies between the two groups (P=0.01 for a 3×2 contingency table). Heterozygosity for the TC genotype was significantly more frequent in cases. FIG. 4C shows genotype distribution for polymorphism at position 1440 in subjects with airflow limitation (Cases) and in those without airflow limitation (Controls). There was a significant difference in genotype frequencies between the two groups (P=0.05 for a 3×2 contingency table). Heterozygosity for the GA genotype was significantly more frequent in cases.

FIGS. 5A-C show genotype distribution based on CXCR2 polymorphisms in Whites. FIG. 5A shows genotype distribution for polymorphism at position 785 in subjects with airflow limitation (Cases) and in those without airflow limitation (Controls) in Whites. There was a significant difference in genotype frequencies between the two groups (P=0.03 for a 3×2 contingency table). Heterozygosity for the C/T genotype was significantly more frequent in cases. FIG. 5B shows genotype distribution for polymorphism at position 1208 in subjects with airflow limitation (Cases) and in those without airflow limitation (Controls) in Whites. There was a significant difference in genotype frequencies between the groups (P=0.03 for a 3×2 contingency table). Heterozygosity for the T/C genotype was significantly more frequent in cases. FIG. 5C shows genotype distribution for polymorphism at position 1440 in subjects with airflow limitation (Cases) and in those without airflow limitation (Controls) in Whites. There was a significant difference in genotype frequencies between the two groups (P=0.03 for a 3×2 contingency table). Heterozygosity for the G/A genotype was significantly more frequent in cases.

FIG. 6 shows distribution of CXCR2 haplotypes at positions 785, 1208 and 1440 in Cases and Controls. There were no significant differences in the haplotype frequencies between cases and controls.

FIGS. 7A-C show distribution of CXCR2 polymorphisms stratified by FEV1. FIG. 7A shows distribution of CXCR2-785 polymorphisms stratified by FEV1 in the Whites and Blacks. There was no significant difference in genotype frequencies between the groups (P=0.19 for a 3×3 contingency table). FIG. 7B shows distribution of CXCR2-1208 polymorphisms stratified by FEV1 in the Whites and Blacks. There was no significant difference in genotype frequencies between the three groups (P=0.18 for a 3×3 contingency table). FIG. 7C shows distribution of CXCR2-1440 polymorphisms stratified by FEV1 in the Whites and Blacks. There was no significant difference in genotype frequencies between the three groups (P=0.40 for a 3×3 contingency table). FIGS. 8A-B show distribution of CXCR2 haplotypes stratified by FEV1. FIG. 8A shows distribution of CXCR2 haplotypes stratified by FEV1 in the Whites. CCA, CTA, TTA and TTG haplotypes were particularly rare in this sample population and are not shown in the figure. FIG. 8B shows distribution of CXCR2 haplotypes stratified by FEV1 in the Blacks. CCA, CTA, TTA and TTG haplotypes were particularly rare in this sample population and are not shown in the figure.

FIG. 9 shows that continuous forced expiratory volume in 1 sec (FEV1) and Forced Expiratory Volume in 1 sec/Forced Vital Capacity (FEV1/FVC) are significantly associated with the polymorphisms of CXCR2 at positions 785, 1208 and 1440.

### DETAILED DESCRIPTION OF THE INVENTION

Fluorescence-based single nucleotide polymorphism detection assays offer several important advantages over traditional PCR approaches used to determine genotype (e.g. sequencing of PCR products and restriction fragment length polymorphism, RFLP). First, restriction fragment length polymorphism analysis can in some instances result in significant false positive rates as a result of incomplete restriction enzyme digestion or the presence of other mutations close to the mutation of interest. Second, fluorescence-based genotyping assays are more amenable to high-throughput screening, as they do not require extensive post-amplification manipulation.

Commonly used fluorescence-based PCR techniques for single nucleotide polymorphism detection include the use of either the nonspecific DNA intercalating dye SYBR Green I or an allele-specific fluorogenic probe (i.e. Taqman). In many instances, the use of SYBR Green I is more cost-effective when applied to haplotype analysis of genes with multiple allelic variants since it does not require the synthesis of numerous allele-specific fluorogenic probes.

In certain embodiments of the present invention, the use of allele-specific primers containing an additional internal mismatch obviates the need for extensive optimization of PCR amplification conditions associated with traditional PCR amplification of specific alleles. Additionally, the mismatch nucleotides in these primers have little effect on specific PCR product yield, but drastically reduce non-specific product yield to undetectable levels.

Current methods for genotyping the chemokine receptor (CXCR2) include PCR amplification followed by sequencing and fluorogenic probe-based PCR assays. The simple, rapid, inexpensive, reproducible, and reliable real-time PCR genotyping methods presented here constitute a significant improvement over current techniques. Using this approach, genotyping results can be obtained within 2 hours of whole blood or tissue procurement. Importantly, these techniques described herein are also generally applicable in laboratories lacking access to real-time PCR equipment because allelic discrimination can be determined using traditional PCR and agarose gel electrophoresis.

Additionally, the present invention also investigated the association between CXCR2 polymorphisms at positions 785, 1208 and 1440 and airflow limitation, pulmonary function tests and the quality of life. The allelic frequencies found in the control group of the present invention were similar to those reported by Renzoni and coworkers. Additionally, it was observed that the distribution of genotype frequency of the polymorphisms at positions 785, 1208 and 1440 in subjects with airflow limitation was significantly different from that of normal subjects. An excess of heterozygosity of all three polymorphisms was also observed in cases compared to controls, which suggested a deleterious effect associated with the heterozygous genotype at all three positions. Further, a significant association between the CXCR2 polymorphisms at positions 785, 1208 and 1440 and the severity of airflow limitation that was represented by FEV1 percent predicted was also observed.

A significant association of continuous FEV1 and FEV1/FVC with the CXCR2 polymorphisms at all three positions was also observed. CXCR2 is located on chromosome 2q. Autosomal genome-wide linkage studies by DeMeo et al. showed robust linkage for pulmonary function on chromosome 2q. Another study by Silverman et al. showed that the early-onset COPD susceptibility locus was present on chromosome 2 and the significant linkage of FEV1/FVC to chromosome 2q suggested that one or more genes were influencing the development of airflow obstruction.

Additionally, a significant association of the longitudinal decline in pulmonary function (FEV1) with CXCR2 genotypes at positions 785, 1208 and 1440 was also observed. Subjects with heterozygous genotypes at all 3 positions had a rapid decline in FEV1, compared to homozygous or nullizygous subject. A similar trend in decline in functional capacity was also observed with other measures of functional capacity like TWOMINSD.

Further, there was a marked interethnic difference in the frequency of these polymorphisms among African-Americans and Caucasians. The interethnic differences in the distribution of CXCR2 receptor polymorphisms demonstrated in the present invention might raise the possibility that genetic factors affecting the expression and function of the CXCR2 receptor may be important determinants of ethnic differences in COPD prevalence and/or severity.

However, no significant association was observed between the haplotypes with the airflow limitation and any of the pulmonary function measures. It is further contemplated that other polymorphisms in either the CXCR1 or CXCR2 receptors could be used to further define the haplotypes in this population. The CXCR2 C785T polymorphism did not result in an amino acid substitution in the protein sequence and CXCR2 T1208C and G1440A lie in the non-coding region. All of these however have the potential of altering mRNA processing, stability or translation. There could be linkage disequilibrium between these polymorphisms and another polymorphism within the CXCR2 gene or in the immediate region. Thus, it is likely that a modification of CXCR2 induced by the described polymorphisms or by a functional polymorphism close to them, might alter the functionality of CXCR2 receptor. There is considerable evidence, which suggests that the genetic factors influence both the pulmonary function and the risk of developing airflow limitation (Wilk, J. B. et al., 2003; Joost, O. et al., 2002; Malhotra, A. et al., 2003; Sandford, A. J. et al., 2001). In summary, the present invention demonstrated that the distributions of CXCR2 polymorphisms are different between the ethnic groups. These polymorphisms might be important in the phenotype modulation of airflow limitation and in determining its severity.

Thus, the present invention is directed to a primer pair for genotyping a single nucleotide polymorphism of human chemokine receptor (CXCR2) gene comprising: an allele specific forward primer and a reverse primer or an allele specific reverse primer and a forward primer. The allele specific forward primer and the allele specific reverse primer is a wild type allele specific primer or a mutant allele specific primer, where the allele specific primer has an additional internal nucleotide mismatch two-three bases from the 3′ terminus. Further, the wild type allele specific forward primer has a nucleotide sequence at the 3′ end at least 85% homologous to SEQ ID No. 1 or the mutant allele specific forward primer has a nucleotide sequence at the 3′ end at least 85% homologous to SEQ ID No. 2, where the homology is within the first 16 base pairs from the 3′ end. Additionally, the wild type allele specific forward primer has a nucleotide sequence of SEQ ID No. 1 or the mutant allele specific forward primer has a nucleotide sequence of SEQ ID No. 2.

Similarly, the wild type allele specific forward primer has a nucleotide sequence at the 3′ end at least 85% homologous to SEQ ID No. 4 or the mutant allele specific forward primer has a nucleotide sequence at the 3′ end at least 85% homologous to SEQ ID No. 5, where the homology is within the first 16 base pairs from the 3′ end. Additionally, the wild type allele specific forward primer has a nucleotide sequence of SEQ ID No. 4 or the mutant allele specific forward primer has a nucleotide sequence of SEQ ID No. 5. Further, the reverse primer has a nucleotide sequence about 90% homologous to SEQ ID No. 3 or to SEQ ID No. 6. Furthermore, the reverse primer has a nucleotide sequence of SEQ ID No. 3 or SEQ ID No. 6.

Alternatively, the wild type allele specific reverse primer has a nucleotide sequence at the 3′ end at least 85% homologous to SEQ ID No. 7 or the mutant allele specific reverse primer has a nucleotide sequence at the 3′ end at least 85% homologous to SEQ ID No. 8, where the homology is within the first 16 base pairs from the 3′ end. Additionally, the wild type allele specific reverse primer has a nucleotide sequence of SEQ ID No. 7 or the mutant allele specific reverse primer has a nucleotide sequence of SEQ ID No. 8. Further, the forward primer has a nucleotide sequence about 90% homologous to SEQ ID No. 9. Furthermore, the forward primer has a nucleotide sequence of SEQ ID No. 9. Specifically, the single nucleotide polymorphism of the human chemokine receptor (CXCR2) gene is a T1208C, a C785T or a G1440A single nucleotide polymorphism.

The present invention is also directed to a method of genotyping a single nucleotide polymorphism of human chemokine receptor (CXCR2) gene, comprising the steps of: extracting DNA from sample of an individual; amplifying the DNA in separate PCR reactions comprising a wild type allele specific forward primer and a reverse primer and a mutant allele specific forward primer and the reverse primer or a wild type allele specific reverse primer and a forward primer and a mutant allele specific reverse primer and the forward primer; and identifying the products of the DNA amplification, where the presence of products amplified by the allele specific forward primers and the reverse primer or the allele specific reverse primers and the forward primer indicate that the individual has the single nucleotide polymorphism of human chemokine receptor (CXCR2) gene.

This method further comprises evaluating clinical significance of the single nucleotide polymorphism of chemokine receptor (CXCR2) gene in individuals with disease. The evaluating step in this method comprises comparing the incidence of the polymorphism in the individuals to the incidence of polymorphism in samples from control individuals who do not have the disease, where increased incidence of the polymorphism in individuals with the disease compared to the control individuals indicates that the polymorphism is clinically significant in the disease. Following the evaluating step, the prevalence, severity and response to treatment in the population of individuals of same ethnicity can be determined by correlating the incidence of the polymorphism with the disease in the population. In general, the individuals have a rheumatoid or a respiratory disease, where the disease involves neutrophil recruitment.

Furthermore, all aspects regarding allele specific primers in general, primers of SEQ ID Nos. 1-3 for genotyping T1208C single nucleotide polymorphism, SEQ ID Nos. 4-6 for genotyping C785T single nucleotide polymorphism and SEQ ID Nos. 7-9 for genotyping G1440A single nucleotide polymorphism is the same as described earlier. Generally, the products of DNA amplification are identified by method selected from the group consisting of real-time fluorescence-based analysis, melt curve analysis and gel electrophoresis.

The present invention is further directed to a kit for genotyping a single nucleotide polymorphism of human chemokine receptor (CXCR2) gene. This gene comprises allele specific forward primers and a reverse primer of allele specific reverse primers and a forward primer. All other aspects regarding these primers is the same as described earlier.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the DNA and methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### EXAMPLE 1

CXCR2 Single Nucleotide Polymorphisms Genotyping

The present example describes real-time PCR assays for the rapid detection of the chemokine receptor single nucleotide polymorphisms T1208C, C785T and G1440A. These methods can be readily applied to investigate the effect of the chemokine receptor polymorphic expression on pathogenesis of and susceptibility to chronic inflammatory conditions involving neutrophil recruitment, especially rheumatoid and respiratory diseases.

### EXAMPLE 2

Primer Design

PCR primers are listed in Table 1. Oligonucleotide primers were designed for both wild type and mutant alleles based on the published human CXCR2 receptor sequence (sequence accession nos M99412 and M73969) using the online program Primer3. Discrimination between wild type and mutant alleles was achieved using PCR amplification of allele-specific primers to prevent non-Watson Crick base pairing (Okimoto & Dodgson, 1996; Sommer et al., 1992; Bottema et al., 1993; Newton et al., 1989). Two forward primers and a common reverse primer were designed based on the nucleotide difference at the 3′ terminal base for allelic discrimination of T1208C and C785T. However, for allelic discrimination of G1440A, two reverse primers and a common forward primer were designed due to high GC content in the region upstream of the single nucleotide polymorphism location.

Briefly, the allelic discrimination for T1208C was achieved by designing two sense primers (1208FW and 1208FM) based on the nucleotide difference (T or C) located at the 3′ terminal base. In order to prevent the amplification of the nonmatching primer, an additional nucleotide mismatch (A to T) located 3 bases from the 3′ termini of the allele specific primers (1208FW and 1208FM) was incorporated. These changes were made to improve the amplification specificity and to prevent the generation of non-specific products, which could otherwise occur by the annealing and extension of the 1208FW primer to non-specific template.

A similar strategy was used to achieve allelic discrimination for C785T and G1440A (Table 1). Oligo Toolkit was used to detect hairpin structures and primer-dimers. Primers were synthesized by Integrated DNA Technologies, Inc. (Coralville, Iowa). Expected amplicon lengths were 198, 208 and 200 base pairs for T1208C, C785T and G1440A, respectively.

### EXAMPLE 3

Real-Time PCR Amplification

Genomic DNA was obtained for 10 African-Americans and 10 Northern Europeans from the Human Genetic Cell Repository, sponsored by the National Institute of General Medical Sciences. The use of Human Genetic Cell Repository samples was approved by the University of Tennessee Institutional Review Board. Polymorphisms were detected by PCR amplification of specific alleles (PASA) on a SmartCycler® (Cepheid, Sunnyvale, Calif.) using SYBR Green I (Molecular Probes, Eugene, Oreg.), a nonspecific double-stranded DNA intercalating fluorescent dye.

Thus, to achieve allelic discrimination between wild type and mutant alleles, two physically separate PCR reactions containing either wild type or mutant-specific primers were performed. All reactions were carried out in a total volume of 25 μL. Reaction conditions were identical for T1208C, C785T and G1440A except where noted. Each reaction mixture contained a 1:12,500 dilution of SYBR Green I nucleic acid gel stain 10,000× in dimethyl sulfoxide (DMSO) (Molecular Probes); 0.2 mmol/l of dATP, dCTP, dGTP, and dTTP mixture; 200 nmol/l of both forward and reverse primers; 1.0 U of Taq DNA polymerase (Promega, Madison, Wis.); 6% dimethyl sulfoxide; 1× SmartCycler additive reagent (a 5× additive reagent containing bovine serum albumin at 1 mg/mL, Trehalose at 750 nmol/l, and Tween-20 at 1% v/v) (Cepheid, Sunnyvale, Calif.), and 10 ng of genomic DNA in 1× PCR buffer (pH 8.3, 10× solution containing 100 mmol/l Tris-HCl, 500 mmol/l KCl, 15 nmol/l MgCl2 and 0.01% gelatin) (Sigma, St. Louis, Mo.).

The amplification program consisted of initial denaturation of 95° C. (5 minutes) followed by 27 cycles of 95° C. (15 seconds), annealing at 60° C. (30 seconds), extension at 72° C. (45 seconds). After amplification, melt analysis was performed by heating the reaction mixture from 60° C. to 95° C. at the rate of 0.2° C./second. A negative control without DNA template was run with every assay to demonstrate the overall specificity. PCR products containing the sites of polymorphism at nucleotide position 1208 and 1440 were generated for sequencing using the sense primer (5′-GGGTTCCTCCCTTCTCTTCA-3′, SEQ ID No: 10) and the antisense primer (5′-TTACAGGCACTCACCACCAC-3′, SEQ ID No: 11). PCR product containing site of polymorphism at nucleotide position 785 was generated for sequencing using the sense primer (5′-ATGCGGGTCATCTTTGCTGT-3′, SEQ ID No: 12) and the antisense primer (5′-TTGAGGCAGCTGTGAAGGAT-3′, SEQ ID No: 13).

### EXAMPLE 4

PCR Product Analysis

The real-time fluorescence signal generated by the nonspecific double-stranded DNA binding dye SYBR Green I was analyzed using the SmartCycler® software. A threshold cycle (Cₜ) was determined for each sample using the exponential growth phase and the baseline signal from fluorescence versus cycle number plots. A sample was deemed positive if fluorescence exceeded the threshold. Threshold fluorescence level was automatically set by the SmartCycler® software. Melting curve analysis was performed by slowly heating DNA fragments in the presence of the dsDNA-specific fluorescent dye SYBR Green I. As the sample is heated, fluorescence rapidly decreases when the melting temperature of a particular fragment is reached. Negative first derivative peaks ([-dF/dT] vs. temperature), which are characteristic of the PCR product melt temperature, were used to identify specific PCR products. Amplification reactions were also routinely checked for the presence of nonspecific products by agarose gel electrophoresis. The genotyping method was validated using direct sequencing (ABI Prism® 3100, Applied Biosystems, Foster City, Calif.) after the PCR products were isolated by QlAquick (Qiagen, Valencia, Calif.).

### EXAMPLE 5

CXCR2 Genotyping Results

Allele-specific primers containing an additional nucleotide mismatch 3 bases from their 3′ termini had little effect on specific PCR product yield. However, nonspecific PCR product yield was drastically reduced to basically undetectable levels. Consequently, PCR conditions were optimized such that the threshold cycle (Cₜ) was exceeded only when specific amplification occurred (i.e., only in the presence of a primer:template match).

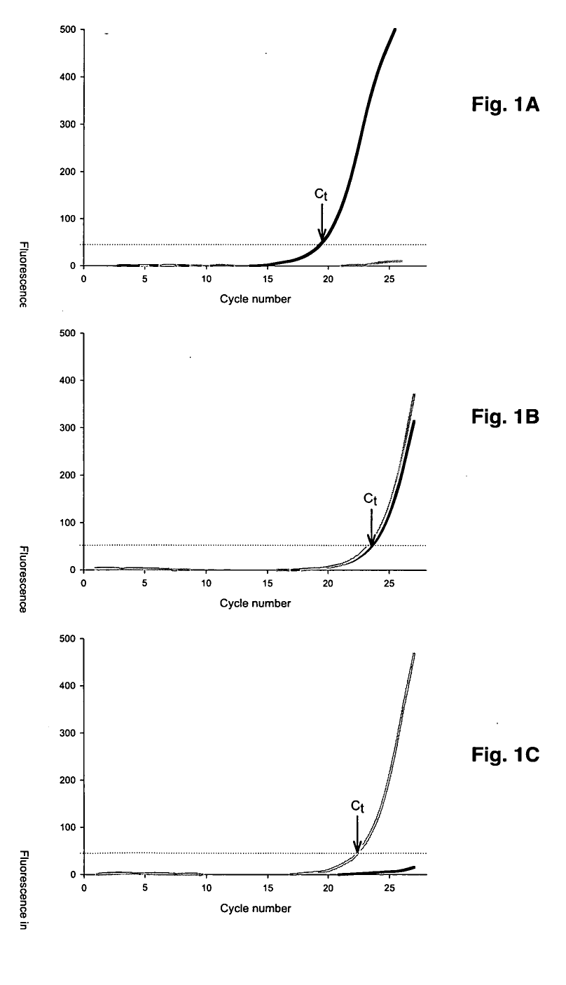

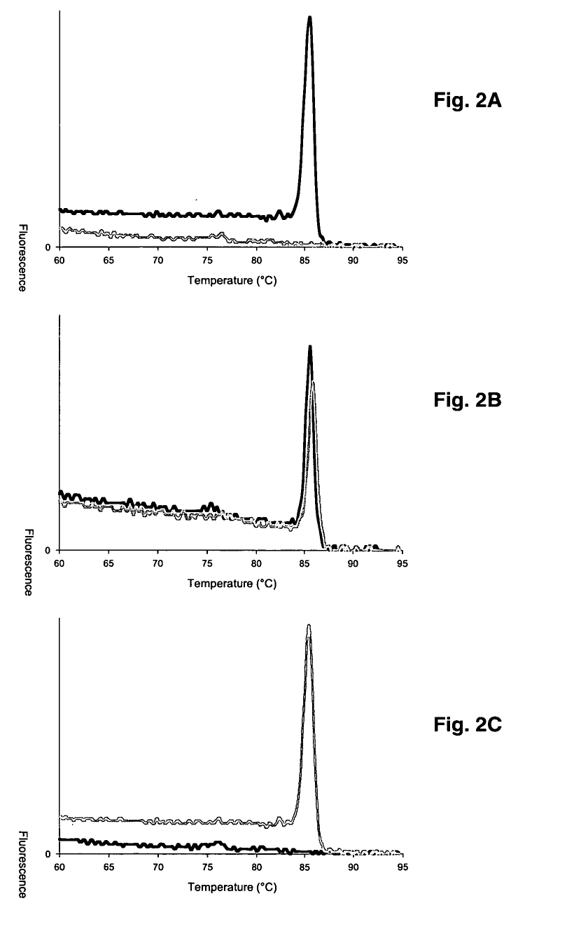

FIG. 1A illustrates the results of the chemokine receptor (CXCR2) T1208C allelic discrimination assay using homozygous 1208T genomic DNA amplified with a common primer 1208R (SEQ ID No. 3) and either the wild-type specific primer 1208FW (SEQ ID No. 1) or the mutant-specific primer 1208FM (SEQ ID No. 2). When primers 1208R and 1208FW were used to amplify homozygous 1208T genomic DNA, the PCR growth curve exceeded the Cₜ value at approximately 21 cycles (FIG. 1A), and the melt analysis (negative first derivative) yielded a characteristic sharp peak at approximately 86° C. for the product (FIG. 2A).

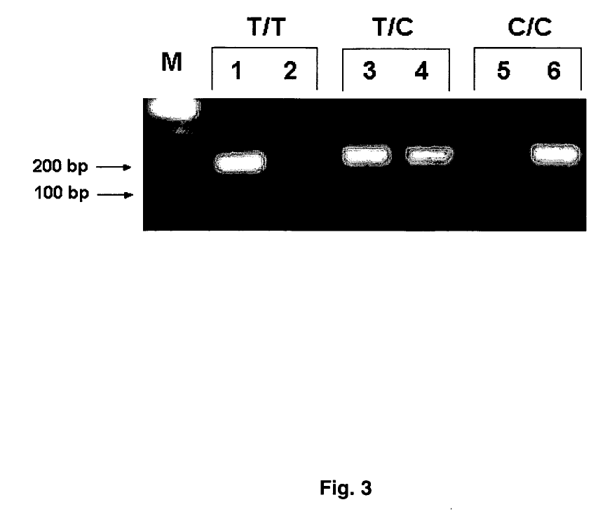

PCR growth curves remained at approximately background fluorescence, and no distinct melt analysis peak was noted when primers 1208R and 1208FM were used to amplify homozygous 1208T genomic DNA (FIGS. 1A and 2). Agarose gel electrophoresis yielded the expected 198-bp fragment when homozygous 1208T DNA was amplified with primers 1208R and 1208FW (FIG. 3). However, no bands were visualized after homozygous 1208T DNA was amplified using primers 1208R and 1208FM (FIG. 3). Similarly, allelic discrimination was achieved after amplification of homozygous 1208C DNA using primers 1208R, 1208FW, and 1208FM (FIGS. 1C and 2C, and 3).

Heterozygous T1208C yielded amplification with both wild type and mutant specific primers and a distinct melt peak was observed after amplification with both wild-type and mutant-specific primers (FIGS. 1B and 2B, and 3). The results were further confirmed by sequencing (data not shown). Results from direct sequencing of selected tested individuals were in perfect agreement with the real-time PCR-based genotyping results. Similarly allelic discrimination was successfully achieved for C785T and G1440A after amplification using their respective primers listed in Table 1.

The genotype frequencies of the sample population of African-Americans and Northern Europeans tested in this study followed Hardy-Weinberg Equilibrium and are presented on Table 2. The observed allele frequencies are similar to those previously reported for Caucasians (Renzoni et al., 2000).

Therefore, a rapid, inexpensive and robust PCR-based screening methodology for chemokine receptor (CXCR2) genotyping, which is a significant improvement over current genotyping techniques with regard to time and starting amount of genomic DNA was developed and validated. Allele specific amplification can also be performed with traditional thermal cyclers followed by agarose gel electrophoresis. This genotyping method can be used to assess the allelic frequency of 1208C, 785T and 1440A in individuals and determine their clinical significance in different populations with various disease states.

TABLE 1
Primer Sequences for CXCR2 Genotyping Based on Accession No. M99412

| | | | |
|---|---|---|---|
| | Annealing | | Product size |
| Polymorphism | Position | Primer Sequence | (bp) |

| | | | | |
|---|---|---|---|---|
| T1208C | 11,061-11,080 | 1208FW-5′ CCATTGTGGTCACAGGATGT 3′ | (SEQ ID NO:1) | 198 |
| | | 1208FM-5′ CCATTGTGGTCACAGGATGC 3′ | (SEQ ID NO:2) | |
| | | 1208R-5′ TGCAGAGCTGTCTCACTGGA 3′ | (SEQ ID NO:3) | |
| C785T | 10,638-10,657 | 785FW-5′ TCGTCCTCATCTTCCTGGTC 3′ | (SEQ ID NO:4) | 208 |
| | | 785FM-5′ TCGTCCTCATCTTCCTGGTT 3′ | (SEQ ID NO:5) | |
| | | 785R-5′ AGTCCATGGCGAAACTTCTG 3′ | (SEQ ID NO:6) | |
| G1440A | 11,338-11,312 | 1440RW-5′ GTATTTTTAGTAGAGACAGGGTTTGAC 3′ | (SEQ ID NO 7) | 200 |
| | | 1440RM-5′ GTATTTTTAGTAGAGACAGGGTTTGAT 3′ | (SEQ ID NO 8) | |
| | | 1440F-5′ CCTCACCCCTTGCCATAAT 3′ | (SEQ ID NO 9) | |

FW and FM: forward primers for wild type and mutant allele, respectively. R indicates reverse primer. F indicates forward primer. RM and RW: reverse primers for wild-type and mutant allele, respectively. Nucleotides in underline indicate the site of polymorphism and corresponding wild type and mutant nucleotides. Nucleotides in bold indicate nucleotide mismatches 3 bases from the 3′ termini of the published sequence, which had little effect on specific PCR product yield but non-specific PCR product yield was drastically reduced to undetectable levels.

TABLE 2
Allele frequency for the CXCR2-SNPs T1208C, C785T and G1440A
in 10 Northern Europeans and 10 African-Americans

| | | | | Genotype frequency | | |
|---|---|---|---|---|---|---|
| | | Allele frequency | | wt/ | wt/ | mut/ |
| SNP | Population | wt | mut | wt | mut | mut |
| T1208C | Northern European | 0.35 | 0.65 | 0.1 | 0.5 | 0.4 |
| | African-American | 0.15 | 0.85 | 0 | 0.3 | 0.7 |
| C785T | Northern European | 0.40 | 0.60 | 0.1 | 0.6 | 0.3 |
| | African-American | 0.25 | 0.75 | 0 | 0.5 | 0.5 |
| G1440A | Northern European | 0.50 | 0.50 | 0.2 | 0.6 | 0.2 |
| | African-American | 0.50 | 0.50 | 0.3 | 0.4 | 0.3 |

Genotyping of chemokine receptor (CXCR2) variants may provide valuable information on disease susceptibility and pathogenesis of common complex disorders that involve neutrophilic inflammatory processes. The CXCR2 C785T polymorphism does not result in an amino acid substitution in the protein sequence and CXCR2 T1208C and G1440A lie in the noncoding region. All of them however, have the potential of altering mRNA processing, stability or translation. Most of the current methods for chemokine receptor (CXCR2) genotyping require post-PCR handling, such as agarose or microtitre array diagonal gel electrophoresis, and thus are time-consuming and may increase the risk of PCR contamination (Kato H. et al., 2000; Renzoni E. et al., 2000). To solve this problem, refined methods are required for rapid and accurate genotyping of the chemokine receptor (CXCR2) gene.

Real-time PCR is an innovative way for detection and quantification of PCR amplification fragments. In the present study, a rapid, inexpensive and robust PCR-based screening methodology for chemokine receptor (CXCR2) genotyping, which is a significant improvement over current genotyping techniques with regard to time and starting amount of genomic DNA was developed and validated. This method takes advantage of the fluorescent property of SYBR Green I dye and the melting curve analysis that allow the detection and distinction of different length of PCR products. The genotyping method used here offers several advantages over traditional PCR approaches used to determine genotype (e.g., sequencing of PCR products and restriction fragment length polymorphism [RFLP]). This method is more amenable to high-throughput screening, as it does not require extensive post amplification manipulation. Also, the use of SYBR Green I is more cost-effective compared to other fluorescence-based PCR techniques for SNP detection.

In addition, the use of allele specific primers containing an additional mismatch eliminates the need for extensive optimization of PCR conditions. Allele specific amplification can also be performed with traditional thermal cyclers followed by agarose gel electrophoresis. This genotyping methodology has been tested and validated for achieving high throughput on ABI Prism® 7000 Sequence Detection System using a 96-well format with the exactly same conditions as the SmartCycler® (Cepheid, Sunnyvale, Calif.). The genotype of every specimen at nucleotide positions 1208, 785, and 1440 was revealed correctly by this real-time PCR assay, indicating the high specificity of this technique.

With no need for post-PCR handling and increased sensitivity using fluorescence chemistry, PCR cycles were reduced to 27 cycles. Less than one hour was needed to complete the setting and performance of 27 PCR cycles, and data analysis. The method is extremely accurate, robust, and can be optimized in a simple and predictable manner. By virtue of its simplicity, the method is versatile and cost-effective with potential for use in industrial scale genetic studies or in the clinical diagnostic setting. This genotyping method will be used to assess the allelic frequency of these alleles and determine their clinical significance in different populations with various disease states. One such use of this method is discussed in the following examples.

### EXAMPLE 6

Study Population

In order to investigate the racial differences in the association between CXCR2 polymorphisms airflow limitation, two hundred and sixty-two subjects (Whites=147, Blacks=115) with airflow limitation (out of 2223 subjects with spirometry data usable for analysis) were selected from the Health ABC study. The design of this study has been described elsewhere (Waterer, G. W. et al., 2001). Study participants were well-functioning subjects aged 70 to 79 in the Memphis, Tenn. and the Pittsburg, Pa. vicinities. These individuals were matched by gender, race and smoking status with 524 subjects of Health ABC with no airflow limitation. Thus, the number of controls was twice the number of individuals with airflow limitation identified in Health ABC at baseline. A biased random sample was obtained for the control group to ensure that the control group contained a similar number of current or former smokers with similar smoking history compared to the COPD group. This allowed analyzing for a gene-environment (smoking) interaction.

Demographics, smoking status and history, respiratory symptoms, medical diagnosis of respiratory disease, medication and pulmonary function test data was obtained from the Health ABC database for these participants. The lung function of the subjects was assessed as FEV1% predicted that is FEV1 adjusted for age, height and sex. Spirometry was performed with a horizontal dry rolling seal spirometer (SensorMedics Corporation, Yorba Linda, Calif.) connected to a personal computer. The individuals with airflow limitation were further divided into the following three groups by their FEV1 values according to GOLD staging system for the severity of the COPD (Ruse, C. E. et al., 2003): FEV1<50% of predicted (severe); FEV1 50 to 79% of predicted (moderate); and FEV1>79% of predicted (mild).

Additionally, spirometry assessments and other measures of functional capacity like Peak Expiratory Flow (PEF), Forced Expiratory Volume in 6 sec (FEV6), Forced Vital Capacity (FVC), FEV1 from the best curve with repetitive measures, FEV1/FVC, ease walking quarter of a mile (EASEQM), ease walking a mile (EASE1M), walking speed (m/sec) over 2 minute (TWOMINSD), walking speed (m/sec) over 400 meters (MTR400SD), Body Mass Index (BMI) and EPESE performance battery score (EPESEPPB) from the Health ABC study subjects at baseline (Year 1) and follow-up. Spirometry was conducted at the Year 1 and Year 5 exams. EASEQM, EASE1M, BMI were monitored at Year 1-6, where as TWOMINSD and MTR400SD were estimated at Years 1, 2, 4 and 6.

### EXAMPLE 7

Genotyping Methods

The discrimination between wild type and mutant alleles was achieved using PCR amplification of allele specific primers (PASA) as described earlier.

### EXAMPLE 8

Statistical Analysis

The frequency distribution of CXCR2 polymorphic genotypes by case-control status was examined by chi-square tests. The association of the polymorphism genotype parameters and the pulmonary function test between healthy subjects and individuals with airflow limitation was examined by the chi-square test for trend in binomial proportions. Deviations from Hardy-Weinberg equilibrium (HWE) were tested by a χ² test of observed and expected cell counts with the degrees of freedom (df) adjusted by the number of independent frequencies estimated. To adjust for additional covariates, the data were reanalyzed by multiple logistic regression with airflow limitation as the outcome variable. For all analyses, the significance level was set at α=0.05. For an identified association, the odds ratio (OR) (and its 95% Cl (Ishii, T. et al., 2000)) was computed as an approximation of the relative risk. The statistical analyses were done applying the SAS 9.0 and the SPLUS 6.0 package.

In Health ABC Study, 262 individuals were identified at baseline as having airflow limitation based spirometry testing. Airflow was analyzed as a continuum from normal to severely abnormal. Although most of the cases have moderate to severe airflow limitation, airflow can also be categorized. Using airflow (either a continuous or categorical variable) as the dependent variable, the genotypic frequencies were compared to examine the existence of evidence for a dose response with respect to a particular polymorphism. General linear model (GLM) was used to test for an association of genotype with continuous FEV1. However, due to small numbers of individuals within some subclasses, dose responses for every race-gender subclass could not be detected. The gene-environment (smoking) interaction was investigated in a subanalysis that included smoking severity and duration.

Haplotype frequencies were estimated using the expectation-maximization (EM) algorithm, as haplotypes could be discerned directly from double heterozygotes. Random effects model (REM) was used to get an insight into the association of CXCR2 genotypes with the nature of longitudinal changes occurring over time in various outcome variables. For each subject, a random intercept and random slope was estimated from the base model. The slopes were calculated as the difference between Year 1 and Year 5 mean pulmonary function estimates divided by the average time between them. This model provided an estimate of the relationship between longitudinal changes in outcome variables and the genotypes, similar to that obtained from multiple regression. Addition of the interaction between genotype and time tested whether the slopes were constant over all levels of the genotype.

### EXAMPLE 9

Results

The characteristics of the subjects are summarized in Table 3. Further, the distribution of polymorphisms at amino acid positions 785, 1208 and 1440 within subjects with airflow limitation (cases) and those without airflow limitation (controls) are shown in Table 4.

TABLE 3
Characteristics of the study subjects

| | Controls (n = 520) | Cases (n = 258) |
|---|---|---|
| Males (%) | 55 | 57.4 |
| Smoking status | | |
| Current Smokers (%) | 56.2 | 55.4 |
| Former Smokers (%) | 26.5 | 28.3 |
| Age (yrs)* | 73.5 ± 2.8 | 73.2 ± 2.9 |
| FEV1 (%)* | 99.4 ± 16.3 | 62.7 ± 18.1 |
| FEV1/FVC (%)* | 97.6 ± 14.6 | 80.9 ± 18.6 |
| PEF (%)* | 100.5 ± 22.0 | 57.9 ± 19.7 |
| Packs (yr⁻¹)* | 26.7 ± 29.0 | 40.4 ± 33.4 |

*All the parameters are represented as Mean ± SD.

TABLE 4
Genotype frequencies (%) in Controls and Cases

| | | Frequency | | | | |
|---|---|---|---|---|---|---|
| Locus | Genotype | Controls | | Cases | | P-value |
| CXCR2-785 | C/C | 98 | (19.0%) | 40 | (15.6%) | 0.05 |
| | C/T | 240 | (46.6%) | 144 | (56.0%) | |
| | T/T | 177 | (34.4%) | 73 | (28.4%) | |
| CXCR2-1208 | T/T | 69 | (13.4%) | 25 | (9.7%) | 0.01 |
| | T/C | 193 | (37.5%) | 124 | (48.3%) | |
| | C/C | 253 | (49.1%) | 108 | (42.0%) | |
| CXCR2-1440 | G/G | 176 | (34.2%) | 78 | (30.4%) | 0.05 |
| | G/A | 233 | (45.2%) | 139 | (54.1%) | |
| | A/A | 106 | (20.6%) | 40 | (15.6%) | |

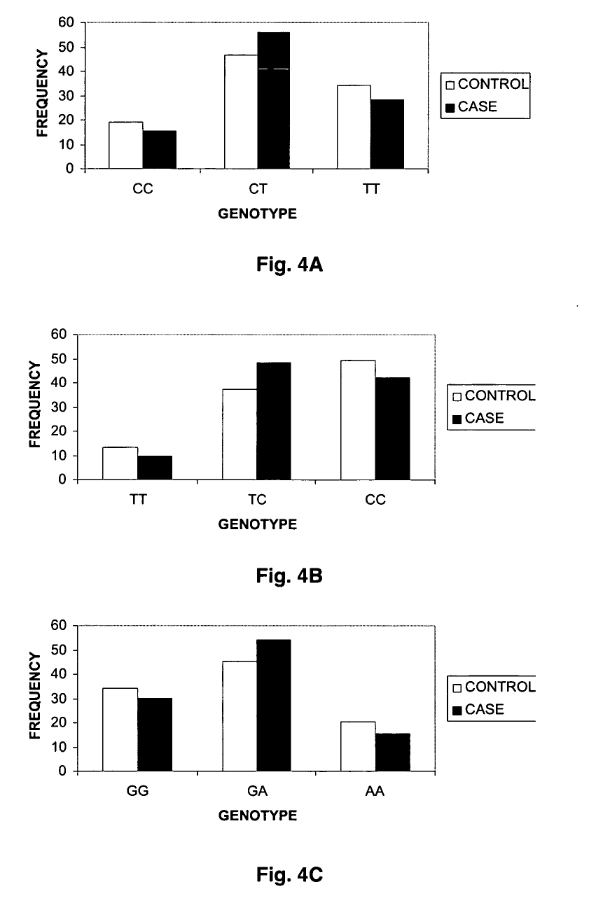

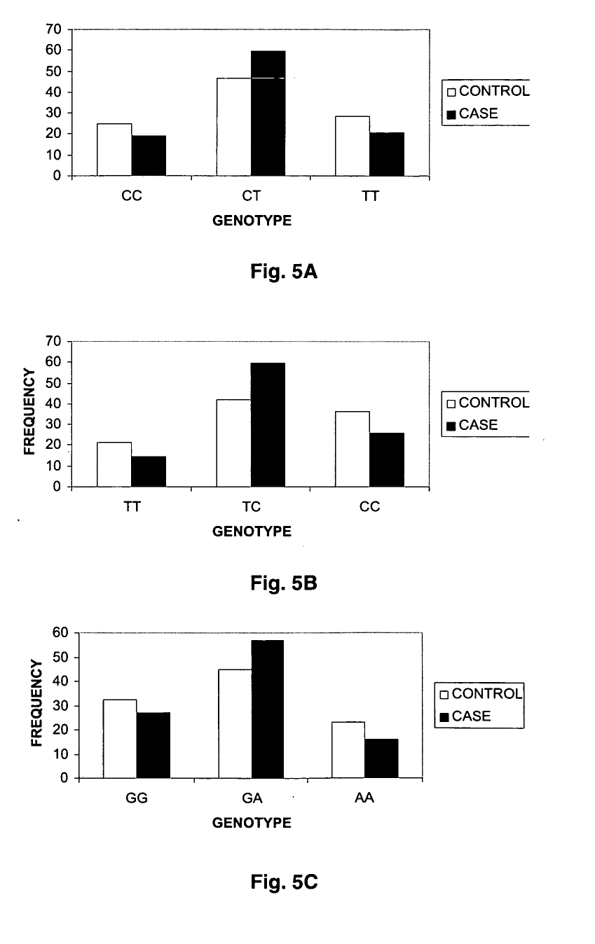

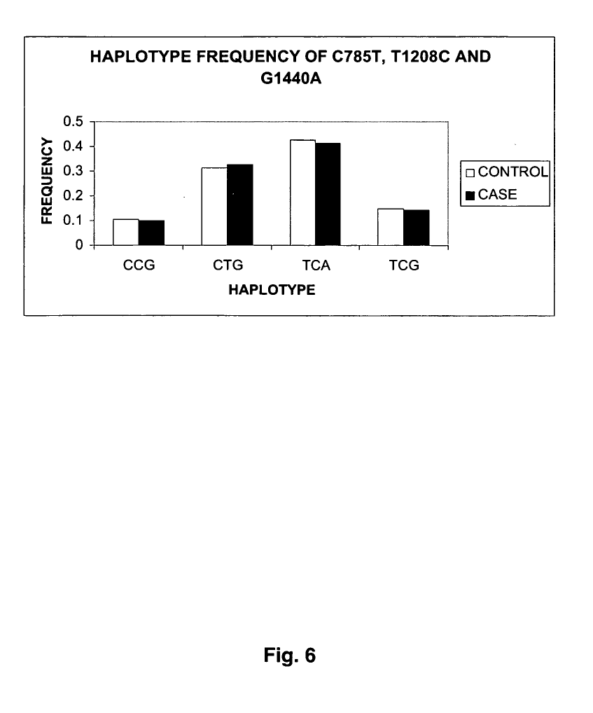

There was significant difference in genotype frequencies between the two groups at the positions 785 (P=0.05), 1208 (P=0.010 and at position 1440 (P=0.05). The frequency of heterozygous C785T (56.0% vs 46.6%), T1208C (48.3% vs 37.5%) and G1440A (54.1% vs 45.2%) was significantly different between the cases and controls, respectively. FIGS. 4A-C show the distribution of genotypes at position 785, 1208 and 1440 within cases and controls. When the genotype frequencies were stratified by race, a significant difference was observed between the cases and controls at position 785, 1208 and 1440 for Whites but no such difference was observed between the two groups for blacks (Table 5). The distribution of genotypes at positions 785, 1208 and 1440 for Whites and Blacks is illustrated in FIGS. 5A-C, respectively. In addition to analyzing individual polymorphisms, the haplotype frequencies were also estimated using the EM algorithm. It was observed that white subjects with a CTG haplotype had a higher FEV1% predicted (P=0.008) compared to subjects with other haplotypes.

TABLE 5
Genotype frequencies (%) in Controls and Cases stratified by race.

| | | Geno- | Frequency | | | |
|---|---|---|---|---|---|---|
| Locus | Race | type | Controls | | Cases | P-value |
| CXCR2-785 | Whites | C/C | 71 | (25%) | 28 | 0.03 |
| | | | | | (19.4%) | |
| | | C/T | 132 | (46.5%) | 86 | |
| | | | | | (59.7%) | |
| | | T/T | 81 | (28.5%) | 30 | |
| | | | | | (20.8%) | |
| | Blacks | C/C | 27 | (11.7%) | 12 | 0.73 |
| | | | | | (10.6%) | |
| | | C/T | 108 | (46.8%) | 58 | |
| | | | | | (51.3%) | |
| | | T/T | 96 | (41.6%) | 43 | |
| | | | | | (38.1%) | |
| CXCR2-1208 | Whites | T/T | 61 | (21.5%) | 21 | 0.003 |
| | | | | | (14.6%) | |
| | | T/C | 120 | (42.3%) | 86 | |
| | | | | | (59.7%) | |
| | | C/C | 103 | (36.3%) | 37 | |
| | | | | | (25.7%) | |
| | Blacks | T/T | 8 | (3.5%) | 4 (3.5%) | 0.93 |
| | | T/C | 73 | (31.6%) | 38 | |
| | | | | | (33.6%) | |
| | | C/C | 150 | (64.9%) | 71 | |
| | | | | | (62.8%) | |
| CXCR2-1440 | Whites | G/G | 92 | (32.4%) | 39 | 0.05 |
| | | | | | (27.1%) | |
| | | G/A | 127 | (44.7%) | 82 | |
| | | | | | (56.9%) | |
| | | A/A | 65 | (22.9%) | 23 | |
| | | | | | (16.0%) | |
| | Blacks | G/G | 84 | (36.4%) | 39 | 0.69 |
| | | | | | (34.5%) | |
| | | G/A | 106 | (45.9%) | 57 | |
| | | | | | (50.4%) | |
| | | A/A | 41 | (17.8%) | 17 | |
| | | | | | (15.0%) | |

A strong linkage disequilibrium was observed between the SNPs at position 785, 1208 and 1440 (P<0.0001). There was no significant difference between allele frequency for polymorphisms at both positions 785, 1208 and 1440 between cases and controls. This data suggested that the heterozygous genotype at all three positions was significantly larger in cases compared to controls and this had a deleterious effect in cases. To ensure that this association was not due to potentially confounding factors, a logistic regression was also performed (Table 6).

TABLE 6
Multiple logistic regressions for cases versus controls

| | Odds | | |
|---|---|---|---|
| Variable | ratio | 95% CI | P-value |
| Heterozygous T1208C genotype | 1.56 | 1.15 to 2.11 | 0.0042 |
| Adjusted Heterozygous T1208C genotype | 1.54 | 1.13 to 2.11 | 0.01 |
| Pack year | 2.82 | 2.05 to 3.88 | <0.0001 |
| (<=40, 0; >40, 1) | | | |

The airflow limitation was examined as main dependent variable relative to genotype. An association between heterozygosity for the polymorphism at position 1208 and airflow limitation remained significant in this analysis. The adjusted odds ratio for the heterozygous T1208C genotype and airflow limitation was 1.54 (95% Cl 1.13 to 2.11, P=0.01). The only other significant covariate was pack year, with an adjusted odds ratio of 2.82 (95% Cl 2.05-3.88, P<0.0001).

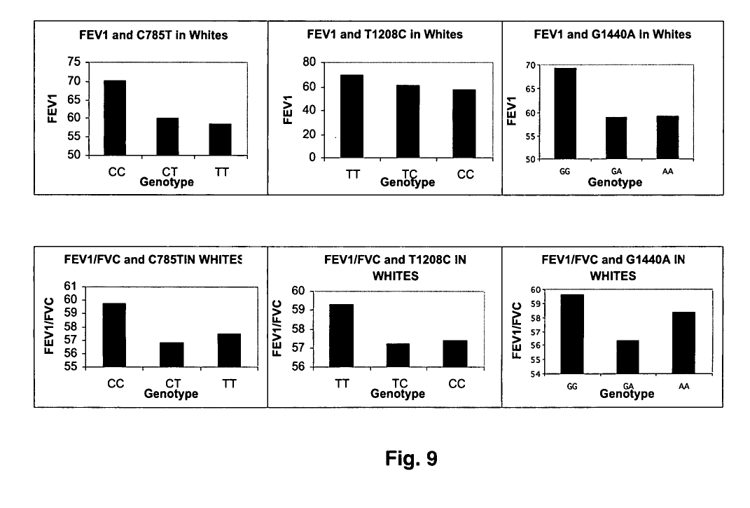

Additionally when the polymorphisms were compared with the results of the patients' FEV1, there was no significant difference at position 785 (P=0.38), 1208 (P=0.10), and 1440 (P=0.15) in both Black and White cases. However, when stratified by race, it was observed that the continuous FEV1 was significantly associated with the polymorphism at position 785 (P=0.009), 1208 (P=0.03) and 1440 (P=0.004) in Whites but was not significant in Blacks (FIG. 9). In Whites, subjects with homozygous C785T, T1208C and G1440A genotypes had a higher FEV1 compared to subjects with either heterozygous or nullizygous genotypes. Similarly, it was also observed that the continuous FEVI/FVC was significantly associated with the polymorphism at position 785 (P=0.01) and 1440 (P=0.06) in Whites but not in Blacks. It was also observed that subjects with homozygous C785T, T1208C and G1440A genotypes had higher FEV1/FVC compared to subjects with either heterozygous or nullizygous genotypes.

The patients were further divided into the following three groups by their FEV1 values according to the proposed GOLD staging system for the severity of the airflow limitation: FEV1<50% of predicted (severe); FEV1 50 to 75% of predicted (moderate); and FEV1>79% of predicted (mild). The genotype frequencies of these polymorphisms were compared in each group (Tables 7 and 8).

TABLE 7
Genotype frequencies (%) stratified by FEV1 in Cases.

| | Geno- | FEV1% predicted | | | | | | |
|---|---|---|---|---|---|---|---|---|
| Locus | type | <50% | | 50-79% | | >79% | | P-value |
| CXCR2- | C/C | 5 | (7.1%) | 24 | (17.5%) | 11 | (22.0%) | 0.19 |
| 785 | C/T | 44 | (62.9%) | 73 | (53.3%) | 27 | (54.0%) | |
| | T/T | 21 | (30.0%) | 40 | (29.2%) | 12 | (24.0%) | |
| CXCR2- | T/T | 4 | (5.7%) | 12 | (8.8%) | 9 | (18.0%) | 0.18 |
| 1208 | T/C | 32 | (45.7%) | 70 | (51.1%) | 22 | (44.0%) | |
| | C/C | 34 | (48.6%) | 55 | (40.2%) | 19 | (38.0%) | |
| CXCR2- | G/G | 19 | (27.1%) | 39 | (28.5%) | 20 | (40.0%) | 0.40 |
| 1440 | G/A | 41 | (58.6%) | 73 | (53.3%) | 25 | (50.0%) | |
| | A/A | 10 | (14.3%) | 25 | (18.3%) | 5 | (10.0%) | |

TABLE 8
Genotype frequencies (%) stratified by FEV1 and race in Cases.

| | | | FEV1% predicted | | | | | | |
|---|---|---|---|---|---|---|---|---|---|
| Locus | Race | Genotype | <50% | | 50-79% | | >79% | | P-value |
| CXCR2-785 | Whites | C/C | 2 | (5.7%) | 17 | (19.5%) | 9 | (40.9%) | 0.02 |
| | | C/T | 23 | (65.7%) | 52 | (59.8%) | 11 | (50.0%) | |
| | | T/T | 10 | (28.6%) | 18 | (20.7%) | 2 | (9.1%) | |
| | Blacks | C/C | 3 | (8.6%) | 7 | (14.0%) | 2 | (7.1%) | 0.51 |
| | | C/T | 21 | (60.0%) | 21 | (42.0%) | 16 | (57.1%) | |
| | | T/T | 11 | (31.4%) | 22 | (44.0%) | 10 | (35.7%) | |
| CXCR2-1208 | Whites | T/T | 3 | (8.6%) | 10 | (11.5%) | 8 | (36.4%) | 0.02 |
| | | T/C | 20 | (57.1%) | 55 | (63.2%) | 11 | (50.0%) | |
| | | C/C | 12 | (34.3%) | 22 | (25.3%) | 3 | (13.6%) | |
| | Blacks | T/T | 1 | (2.9%) | 2 | (4.0%) | 1 | (3.6%) | 0.95 |
| | | T/C | 12 | (34.3%) | 15 | (30.0%) | 11 | (39.3%) | |
| | | C/C | 22 | (62.9%) | 33 | (66.0%) | 16 | (57.1%) | |
| CXCR2-1440 | Whites | G/G | 4 | (11.4%) | 24 | (27.6%) | 11 | (50.0%) | 0.03 |
| | | G/A | 24 | (68.6%) | 48 | (55.2%) | 10 | (45.5%) | |
| | | A/A | 7 | (20.0%) | 15 | (17.2%) | 1 | (4.6%) | |
| | Blacks | G/G | 15 | (42.9%) | 15 | (30.0%) | 9 | (32.1%) | 0.59 |
| | | G/A | 17 | (48.6%) | 25 | (50.0%) | 15 | (53.6%) | |
| | | A/A | 3 | (8.6%) | 10 | (20.0%) | 4 | (14.3%) | |

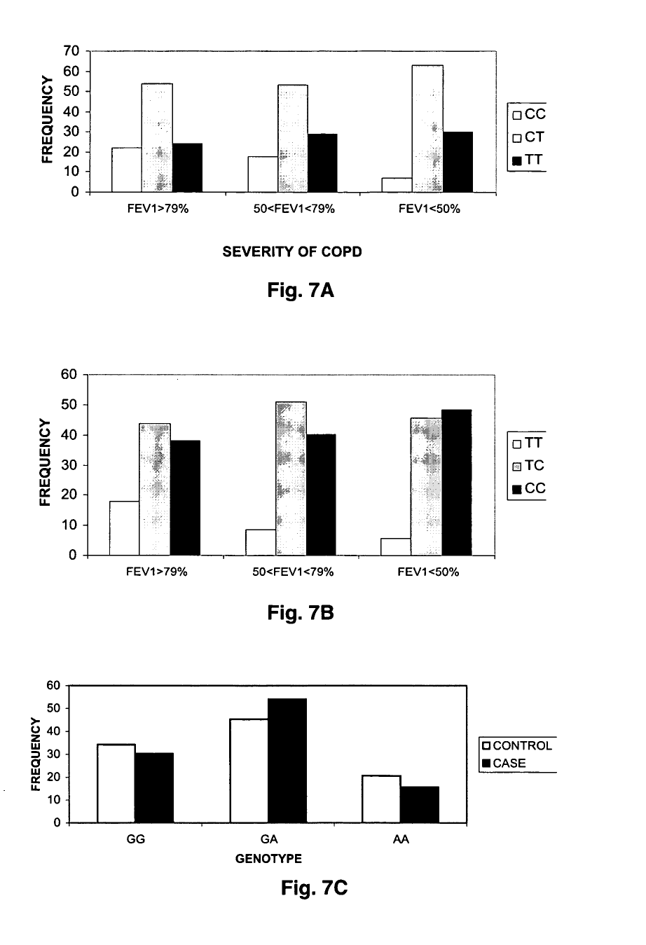

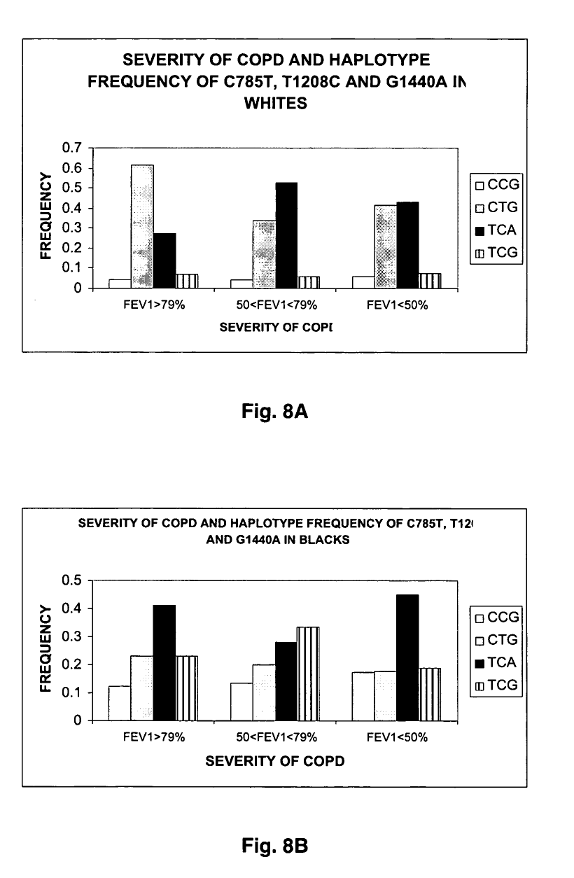

There was no significant correlation of any of the polymorphisms with FEV1 percent predicted when analyzed for both Black and White cases (FIGS. 7A-C). When stratified by race in cases, it was observed that FEV1 percent predicted significantly correlated with all the three polymorphisms at position 785, 1208 and 1440 only in Whites but not in Blacks respectively (FIGS. 8A-B). It was also observed that the severity of COPD significantly associated with the haplotype of CXCR2 polymorphisms at positions 785, 1208 and 1440 in Whites (FIG. 9).

There was significant association of PEF with the G1440 in Whites. Subjects with homozygous G1440A polymorphism had a higher PEF compared to subjects with either heterozygous or nullizygous mutation. No significant association of PEF was observed with the other two SNPs at positions 785 and 1208. There was no significant association observed between FEV6 data in cases and any of the three polymorphisms at position 785, 1208 and 1440 respectively. There was no other significant correlation between any of the CXCR2 polymorphisms and other parameters measured in the pulmonary function test.

Secondary analysis was performed for measures of functional capacity and quality of life associated with the pulmonary disease. There was no significant association between any of the three polymorphisms and the quality of life (FPHSTAT), which was assessed by a question regarding health in general. There was no significant association between the polymorphisms at position 785, 1208 and 1440 and other measures of functional capacity like ease EASEQM, EASE1M, MTR400SD, TWOMINSD, BMI AND EPESEPPB. No significant difference was observed with respect to the center of patient recruitment between the two vicinities. Additionally, no significant effect of gender was observed on any of the analysis.

The longitudinal results in pulmonary function measures (FEV1) after 5 years of follow-up showed an association with CXCR2 genotypes at positions 785, 1208 and 1440. More rapid decliners (Year 1-Year 5) in FEV1 were observed in White subjects with heterozygous genotypes at C785T, T1208C and G1440A. No conclusive association of FEV1 decline with any of the genotypes in Blacks was observed However, more rapid decliners (Year 1-Year 5) in TWOMINSD were observed in subjects with heterozygous genotypes at positions 785, 1208 and 1440 compared to subjects with the homozygous or nullizygous genotypes at all three positions. Due to the physical limitation of these subjects, TWOMINSD might be the best measure of decline in functional capacity in these subjects. Further, EASE1M and EASEQM were both categorical variables and did not change significantly at the follow-up.

The following references were cited herein:
Ahuja S. K. et al. (1994) J Biol Chem 269(42): 26381-26389.
Baggiolini M. (1998) Nature 392(6676): 565-568.
Barnes P. J. (1999) Thorax 54(3):245-252.
Benetazzo M. G. et al. (1999) Respir Med 93(9): 648-654.
Bottema et al. (1993) Methods Enzymol 218:388-402.
Cheng S. L. et al. (2004) Eur Respir J 23(6): 818-824.
Dialyna I. A. et al. (2003) Oncol Rep 10(6): 1829-1835.
Hamajima N. et al. (2002) J Epidemiol 12(3):229-236.
Harrison D. J et al (1997) Hum Exp Toxicol 16(7): 356-360.
Higham M. A. et al. (2000) Eur Respir J 15(2): 281-284.
Holmes W. E. et al. (1991) Science 253(5025): 1278-1280.
Ho L. I. et al. (2001) Chest 120(5): 1493-1499.
Ishii T. et al. (2000) Eur J Clin Invest 30(6): 543-548.
Ishii T. et al. (1999) Thorax 54(8): 693-696.
Joos L. et al. (2002) Hum Mol Genet 11 (5): 569-576.
Joos L. et al. (2003) Thorax 58(8): 703-707.
Joost et al. (2002) Am J Respir Crit Care 165(6): 556-561.
Kato H. et al. (2000) Genes Immun 1(5):330-337.
Keatings V. M. et al. (2000) Chest 118(4): 971-975.
Laufs J. et al. (2004) Am J Pharmacogenomics 4(1): 63-68.
Malhotra et al. (2003) Am J Respir Crit Care 168(5): 556-561.
Mollereau C. et al. (1993) Genomics 16(1): 248-251.
Morris S. W. et al. (1992) Genomics 14(3): 685-691.
Minematsu et al. (2001) Biochem Biophys Res Commun 289(1): 116-119.
Newton et al. (1989) Nucleic Acids Res 17(7):2503-2516.
Okimoto et al. (1996) Biotechniques 21(1):20-22, 24, 26.
Poller, W. et al. (1990) Eur J Clin Invest 20(1): 1-7.
Poller, W. et al. (1993) Genomics 17(3): 740-743.
Renzoni E. et al. (2000) Arthritis Rheum 43(7):1633-1640.
Rodriguez F. et al. (2002) Anal Biochem 308(1): 120-126.
Ruse C. E. et al. (2003) J Am Geriatr Soc 51(9): 1265-1269.
Sandford A. J. et al. (1998) Dis Markers 13(4): 257-260.
Sandford et al. (2001) Am J Respir Crit Care Med 163(2): 469-473.
Sakao et al. (2001) Am J Respir Crit Care Med 163(2): 420-422.
Schellenberg et al. (1998) Am J Respir Crit Care 157 (3 pt 1): 957-961.
Sommer S. S. et al. (1992) Biotechniques 12(1): 82-7.
Sprenger H. et al. (1994) J Biol Chem 269(15): 11065-11072.
Teramoto S. and Ishii T. (2001) Chest 119(1): 315-316.
van der Pouw Kraan et al. (2002) Genes Immun 3(7): 436-439.
Waterer G. W. et al. (2001) J Am Geriatr Soc 49(8): 1032-1038.
Wilk et al. (2003) Am J Respir Crit Care Med 167(11): 1528-1533.
Yim J. J. et al. (2000) Thorax 55(2): 121-125.
Yim J. J. et al. (2002) Lung 180(2): 119-125.
Yoshikawa M. et al. (2000) Int J Mol Med 5(1): 49-53.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

1. A primer pair for genotyping a single nucleotide polymorphism of human chemokine receptor (CXCR2) gene, comprising:
an allele specific forward primer and a reverse primer; or
an allele specific reverse primer and a forward primer.

2. The primer pair of claim 1, wherein said allele specific forward primer and said allele specific reverse primer is a wild type allele specific primer or a mutant allele specific primer, wherein said allele specific primer has an additional internal nucleotide mismatch two-three bases from the 3′ terminus.

3. The primer pair of claim 2, wherein said wild type allele specific forward primer has a nucleotide sequence at the 3′ end at least 85% homologous to SEQ ID No. 1 or said mutant allele specific forward primer has a nucleotide sequence at the 3′ end at least 85% homologous to SEQ ID No. 2, wherein said homology is within first 16 base pairs from the 3′ end.

4. The primer pair of claim 2, wherein said wild type allele specific forward primer has a nucleotide sequence of SEQ ID No. 1 or said mutant allele specific forward primer has a nucleotide sequence of SEQ ID No. 2.

5. The primer pair of claim 2, wherein said wild type allele specific forward primer has a nucleotide sequence at the 3′ end at least 85% homologous to SEQ ID No. 4 or said mutant allele specific forward primer has a nucleotide sequence at the 3′ end at least 85% homologous to SEQ ID No. 5, wherein said homology is within the first 16 base pairs from the 3′ end.

6. The primer pair of claim 2, wherein said wild type allele specific forward primer has a nucleotide sequence of SEQ ID No. 4 or said mutant allele specific forward primer has a nucleotide sequence of SEQ ID No. 5.

7. The primer pair of claim 1, wherein said reverse primer has a nucleotide sequence about 90% homologous to SEQ ID No.: 3 or to SEQ ID No. 6.

8. The primer pair of claim 1, wherein said reverse primer has a nucleotide sequence of SEQ ID No.: 3 or SEQ ID No. 6.

9. The primer pair of claim 2, wherein said wild type allele specific reverse primer has a nucleotide sequence at the 3′ end at least 85% homologous to SEQ ID No. 7 or said mutant allele specific reverse primer has a nucleotide sequence at the 3′ end at least 85% homologous to SEQ ID No. 8, wherein said homology is within first 16 base pairs from the 3′ end.

10. The primer pair of claim 2, wherein said wild type allele specific reverse primer has a nucleotide sequence of SEQ ID No. 7 or said mutant allele specific reverse primer has a nucleotide sequence of SEQ ID No. 8.

11. The primer pair of claim 1, wherein said forward primer has a nucleotide sequence about 90% homologous to SEQ ID No.: 9.

12. The primer pair of claim 1, wherein said forward primer has a nucleotide sequence of SEQ ID No.: 9.

13. The primer pair of claim 1, wherein said single nucleotide polymorphism of the human chemokine receptor (CXCR2) gene is a T1208C, a C785T or a G1440A single nucleotide polymorphism.

14. A method of genotyping a single nucleotide polymorphism of human chemokine receptor (CXCR2) gene, comprising the steps of:
extracting DNA from sample of an individual;
amplifying said DNA in separate PCR reactions comprising a wild type allele specific forward primer and a reverse primer and a mutant allele specific forward primer and the reverse primer or a wild type allele specific reverse primer and a forward primer and a mutant allele specific reverse primer and the forward primer; and
identifying the products of said DNA amplification, wherein the presence of products amplified by said allele specific forward primers and said reverse primer or said allele specific reverse primers and said forward primer indicate that the individual has said single nucleotide polymorphism of human chemokine receptor (CXCR2) gene.

15. The method of claim 14, further comprising:
evaluating clinical significance of said single nucleotide polymorphism of chemokine receptor (CXCR2) gene in individuals with disease.

16. The method of claim 15, wherein said evaluating step comprises:
comparing the incidence of the polymorphism in said individuals to the incidence of the polymorphism in samples from control individuals who do not have the disease, wherein increased incidence of the polymorphism in individuals with said disease compared to said control individuals indicates that said polymorphism is clinically significant in said disease.

17. The method of claim 15, further comprising:
correlating the incidence of said polymorphism with said disease in the population of individuals of same ethnicity to determine prevalence, severity and response to treatment in said population.

18. The method of claim 15, wherein said disease is a rheumatoid or a respiratory disease, wherein said disease involves neutrophil recruitment.

19. The method of claim 14, wherein said wild type or said mutant allele specific primers have an additional internal nucleotide mismatch two-three bases from the 3′ terminus.

20. The method of claim 14, wherein said single nucleotide polymorphism of human chemokine receptor (CXCR2) gene is a T1208C, a C785T or a G1440A single nucleotide polymorphism.

21. The method of claim 20, wherein primers for genotyping said T1208C single nucleotide polymorphism comprises a wild type allele specific forward primer having a nucleotide sequence at the 3′ end at least 85% homologous to SEQ ID No. 1, a mutant allele specific forward primer having a nucleotide sequence at the 3′ end at least 85% homologous to SEQ ID No. 2 and a reverse primer having a nucleotide sequence about 90% homologous to SEQ ID No. 3, wherein said homology at the 3′ end is within the first 16 base pairs.

22. The method of claim 20, wherein primers for genotyping said T1208C single nucleotide polymorphism comprises a wild type allele specific forward primer having a nucleotide sequence of SEQ ID No. 1, a mutant allele specific forward primer having a nucleotide sequence of SEQ ID No. 2 and a reverse primer having a nucleotide sequence of SEQ ID No. 3.

23. The method of claim 20, wherein primers for genotyping said C785T single nucleotide polymorphism comprises a wild type allele specific forward primer having a nucleotide sequence at the 3′ end at least 85% homologous to SEQ ID No. 4, a mutant allele specific forward primer having a nucleotide sequence at the 3′ end at least 85% homologous to SEQ ID No. 5 and a reverse primer with a nucleotide sequence about 90% homologous to SEQ ID No. 6, wherein said homology at the 3′ end is within the first 16 base pairs.

24. The method of claim 20, wherein the primers for genotyping said C785T single nucleotide polymorphism comprises a wild type allele specific forward primer having a nucleotide sequence of SEQ ID No. 4, a mutant allele specific forward primer having a nucleotide sequence of SEQ ID No. 5 and a reverse primer having a nucleotide sequence of SEQ ID No. 6.

25. The method of claim 20, wherein primers for genotyping said G1440A single nucleotide polymorphism comprises a wild type allele specific reverse primer having a nucleotide sequence at the 3′ end at least 85% homologous to SEQ ID No. 7, a mutant allele specific reverse primer having a nucleotide sequence at the 3′ end at least 85% homologous to SEQ ID No. 8 and a forward primer with a nucleotide sequence about 90% homologous to SEQ ID No. 9, wherein said homology at the 3′ end is within the first 16 base pairs.

26. The method of claim 20, wherein the primers for genotyping said G1440A single nucleotide polymorphism comprises a wild type allele specific reverse primer having a nucleotide sequence of SEQ ID No. 7, a mutant allele specific reverse primer having a nucleotide sequence of SEQ ID No. 8 and a forward primer having a nucleotide sequence of SEQ ID No. 9.

27. The method of claim 14, wherein the identification of a product having 198 base pairs corresponds to a product with T1208C, the identification of a product having 208 base pairs corresponds to a product with C785T and the identification of a product having 200 base pairs corresponds to a product with G1440A single nucleotide polymorphisms of human chemokine receptor (CXCR2) gene.

28. A kit for genotyping a single nucleotide polymorphism of human chemokine receptor (CXCR2) gene, comprising:
allele specific forward primers and a reverse primer; or
allele specific reverse primers and a forward primer; and

29. The kit of claim 28, wherein said allele specific primers are wild type allele specific primers or mutant allele specific primers, wherein said allele specific primers have an internal nucleotide mismatch two-three bases from the 3′ terminus.

30. The kit of claim 28, wherein said single nucleotide polymorphism of human chemokine receptor (CXCR2) gene is a T1208C, a C785T or a G1440A single nucleotide polymorphism.

31. The kit of claim 30, wherein primers for genotyping said T1208C single nucleotide polymorphism comprises a wild type allele specific forward primer having nucleotide sequence at the 3′ end at least 85% homologous to SEQ ID No. 1, a mutant allele specific forward primer having nucleotide sequence at the 3′ end at least 85% homologous to SEQ ID No. 2 and a reverse primer having nucleotide sequence about 90% homologous to SEQ ID No. 3, wherein the homology at the 3′ end is within first 16 base pairs.

32. The kit of claim 30, wherein the primers for genotyping said T1208C single nucleotide polymorphism comprises a wild type allele specific forward primer having a nucleotide sequence of SEQ ID No. 1, a mutant allele specific forward primer having a nucleotide sequence of SEQ ID No. 2 and a reverse primer having a nucleotide sequence of SEQ ID No. 3.

33. The kit of claim 30, wherein the primers for genotyping said C785T single nucleotide polymorphism comprises a wild type allele specific forward primer having a nucleotide sequence at the 3′ end at least 85% homologous to SEQ ID No. 4, a mutant allele specific forward primer having a nucleotide sequence at the 3′ end at least 85% homologous to SEQ ID No. 5 and a reverse primer having a nucleotide sequence about 90% homologous to SEQ ID No. 6, wherein said homology at the 3′ end is within first 16 base pairs.

34. The kit of claim 30, wherein the primers for genotyping said C785T single nucleotide polymorphism comprises a wild type allele specific forward primer having a nucleotide sequence of SEQ ID No. 4, a mutant allele specific forward primer having a nucleotide sequence of SEQ ID No. 5 and a reverse primer having a nucleotide sequence of SEQ ID No. 6.

35. The kit of claim 30, wherein the primers for genotyping said G1440A single nucleotide polymorphism comprises a wild type allele specific reverse primer having a nucleotide sequence at the 3′ end at least 85% homologous to SEQ ID No. 7, a mutant allele specific reverse primer having a nucleotide sequence at the 3′ end at least 85% homologous to SEQ ID No. 8 and a forward primer having a nucleotide sequence about 90% homologous to SEQ ID No. 9, wherein said homology at the 3′ end is within first 16 base pairs.

36. The kit of claim 30, wherein the primers for genotyping said G1440A single nucleotide polymorphism comprises a wild type allele specific reverse primer having a nucleotide sequence of SEQ ID No. 7, a mutant allele specific reverse primer having a nucleotide sequence of SEQ ID No. 8 and a forward primer having a nucleotide sequence of SEQ ID No. 9.
